(19) United States
(12) Patent Application Publication
(10) Pub. No.: US 2006/0122655 A1
(43) Pub. Date: Jun. 8, 2006

(54) High-energy battery power source with low internal self-discharge for implantable medical use

(76) Inventor: Wilson Greatbatch, Williamsville, NY (US)

Correspondence Address: Walter W. Duft, Suite 2, 8616 Main Street, Williamsville, NY, 14221, US

(21) Appl. No.: 11/005,304

(22) Filed: Dec. 2, 2004

(51) Int. Cl. A61N 1/372

(52) U.S. Cl. 607/33

(57) ABSTRACT

A high-energy power source with low internal self discharge for implantable use includes a multiplicity of rechargeable energy storage battery cells, a primary power source adapted to charge the energy storage cells, a switching system adapted to switch the energy storage cells between a parallel connection configuration for charging and a series connection configuration for discharging, and circuitry adapted to initiate charging of the energy storage cells only in response to an input signifying a need to discharge energy and to refrain from charging the energy storage cells until the input is received. In this way, the energy storage cells are maintained in a low charge state until discharge energy is required, the low charge state being at a level that promotes low internal self-discharge of the energy storage cells.

### CROSS-REFERENCE TO RELATED APPLICATIONS

This application claims benefit of the tiling date of U.S. Provisional Application No. 60/______, filed on Nov. 30, 2004.

### BACKGROUND OF THE INVENTION

1. Field of the Invention

The present invention relates to improvements in the performance of implantable defibrillators, ICDs (Implantable Cardioverter-Defibrillators) and other battery-powered implantable medical devices designed to provide high-energy electrical stimulation of body tissue for therapeutic purposes.

2. Description of Prior Art

High-energy battery powered medical devices, such as implantable defibrillators and ICDs, are designed to deliver a strong electrical shock to the heart when called upon to correct an onset of tachyarrhythmia. In traditional devices of this type, the energy pulse is produced by charging one or more high-voltage energy storage capacitors from a low voltage battery and then rapidly discharging the capacitors to deliver the intended therapy. This concept is widely practiced and disclosed in numerous patents, including U.S. Pat. No. 4,475,551 of Mirowski dated Oct. 9, 1984. Additionally, much clinical data on defibrillation therapy has been collected and published. See, for example, Gregory P. Walcott, et al. “Mechanisms of Defibrillation for Monophasic and Biphasic Waveforms,” Pacing and Clinical Electrophysiology, March 1994:478; and Andrea Natale, et al. “Comparison of Biphasic and Monophasic Pulses,” Pacing and Clinical Electrophysiology, July 1995:1354.

One of the fundamental components required for a high-energy therapy system is one or more high-voltage energy storage capacitors. Components suitable for implantable medical applications must provide extreme reliability, good electrical performance, small volume and preferably, a low unit cost. In the past, the best compromise solution for these requirements has been the aluminum oxide electrolytic capacitor. Aluminum electrolytic capacitors have been manufactured for many years and have delivered acceptable reliability and performance. There are drawbacks with aluminum electrolytic capacitors however that limit the performance of newer devices in which they are utilized. Among these limitations are form factor, energy storage efficiency and the need for periodic reformation.

The most common construction for aluminum electrolytic capacitors is a “jelly roll” form wherein long strips of aluminum foils and a dielectric layer are stacked and then rolled into a cylindrical form. Two capacitors of this construction are typically required for an ICD device because the fundamental limitation of maximum voltage for an aluminum electrolytic capacitor is only one-half of the potential necessary (700-800 VDC) to deliver the level of energy typically needed to arrest fibrillation. These capacitors are not ideal for the small volume requirements of implantable devices because of the difficulty in packaging other rectilinear components adjacent to the cylindrical capacitors. A number of efforts have been made to replace the cylindrical capacitors with alternative dielectric materials and form factors. Examples of this prior art can be found in U.S. Pat. No. 5,131,388 of Pless, et al. and U.S. Pat. No. 6,009,348 of Rorvick, et al.

In the prior art disclosed by Pless, et al. the energy storage capacitors are of aluminum electrolytic construction but the electrode/separator structure consists of multiple stacked layers rather than a single wound strip. This construction yields a rectilinear form factor whose outline may be optimized to reduce the overall device volume. Pless also discloses a polymeric package for the capacitor(s) in addition to the more traditional aluminum case. This prior art fails to address the significant issue of maintaining intimate contact between the anode, separator and cathode layers of the capacitor. Because the capacitance of any anode/cathode pair is inversely proportional to the spacing between the electrodes (C=* Area/Distance), it is imperative that the spacing for each pair of electrodes be stringently maintained. The need to maintain tight spacing is inherently satisfied by the traditional wrapped cylindrical construction, but Pless fails to disclose how the electrode spacing will be maintained in the planar stacked configuration. Pless also fails to disclose any means for fabricating the electrode foils and subsequently stacking them while maintaining registration for the individual anodes and cathodes.

A number of methods and processes for construction of a flat or rectangular capacitor suitable for a defibrillator or ICD are disclosed in detail in U.S. Pat. No. 6,009,348 of Rorvick, et al. This prior art addresses many of the requirements to reduce the art to practice, including fabrication of the electrode foils, registration of the electrode layers, application and termination of electrical connections and impregnation of the electrolyte into the completed structure. While this disclosure mitigates the shortcomings of the traditional cylindrical capacitors, the device performance is still limited by the fundamental performance of the aluminum electrolytic capacitor.

Representative aluminum capacitors typically provide an energy storage density of 1.5 to 2.0 joules per cm³ so that a defibrillators or ICD specified to deliver 30 joules of defibrillation energy will require a volume of 15 to 20 cm³ for the energy storage capacitors. This represents at least 50% of the available internal volume for a device with an overall volume of 33 cm³ which is typical for modern devices.

More recently, capacitors utilizing tantalum as the dielectric material have been developed for implantable medical applications. These components provide improved volumetric efficiency over traditional aluminum electrolytic capacitors, typically 6 joules per cm³ vs. 2 joules per cm³, but at a higher component cost. A number of embodiments of this newer technology are disclosed in U.S. Pat. No. 6,334,879 of Muffoletto, et al.

Common to both aluminum and tantalum capacitors are two additional shortcomings that limit the overall performance of devices in which they are used. The first of these limitations is the need to periodically reform the dielectric material and the second is the inefficiency of the capacitor as an energy storage device due to dielectric absorption. When electrolytic capacitors are used for energy storage in a defibrillation application, the capacitors will be idle in a non-charged state for the vast majority of their service life. If defibrillation is required the capacitors will be charged in less than 20 seconds and rapidly discharged to deliver therapy to the patient. As long as the patient does not require therapy the capacitors would not normally be charged.

Unfortunately, it is well known by those familiar with aluminum electrolytic capacitors that the energy storage capability and leakage current of these capacitors will degrade over time when the capacitor is not charged. This degradation is attributed to deterioration of the aluminum oxide film and can be reversed by charging the capacitor. When wet-tantalum capacitors were first applied to defibrillator/ICD applications, it was widely believed that they would not be subject to degradation during long idle periods. In U.S. Pat. No. 6,761,728 of Harguth, et al., the need for periodic reforming of wet-tantalum capacitors is identified and a method for achieving periodic reforming in a device is disclosed.

Because the capacitors must be available to perform as specified whenever the patient requires therapy, defibrillators and ICD devices are configured to periodically charge and discharge the energy storage capacitors to reform the dielectric layer in the absence of defibrillation cycles which would also reform the capacitors. This periodic reforming process draws and dissipates energy from the device battery that would otherwise be available for supporting the operation of the device. The need to reform the capacitors is therefore undesirable because it uses battery energy that provides no direct benefit to the patient.

With respect to the second shortcoming, which is storage efficiency, both aluminum and tantalum capacitors suffer from dielectric absorption wherein a portion of the electrical charge imparted to the capacitor will be absorbed in the dielectric material. When an electrolytic capacitor is rapidly discharged as is the case in a defibrillator application, the absorbed charge is not immediately released in the discharge process. If the voltage on the capacitor is monitored after the initial discharge load is removed it will be observed to build, indicating that a portion of the energy stored in the capacitor still remains after the discharge pulse. The magnitude of dielectric absorption is proportional to the dielectric constant of the capacitive material utilized, so that materials with high dielectric constants that will yield the highest capacitance per unit volume will also result in capacitors with the highest dielectric absorption and therefore the lowest energy storage efficiency in a pulse discharge application. Capacitors designed for defibrillator applications will typically deliver only 80% to 90% of the energy on discharge that was instilled to them during charging. Again, primary battery energy that could otherwise provide a benefit to the patient will be lost, making the defibrillator or ICD less efficient.

As an alternative to using high-energy capacitors in implantable devices, U.S. Pat. No. 5,369,351 of Adams and U.S. Pat. No. 6,782,290 of Schmidt propose the use of charge storage arrays based on batteries. However, neither patent provides information regarding the internal self-discharge characteristics of the disclosed energy storage arrays, which are important when determining recharge requirements. Nor do these patents address how internal self-discharge can be minimized to an acceptable level so as not to drain the primary battery power source used to charge the energy storage batteries.

It is to improvements in the practical design of high-energy implantable devices that the present invention is concerned. In particular, the invention is directed to a high-energy power source for use in an implantable defibrillator, ICD or other battery-powered medical device, and which has low internal self-discharge.

### SUMMARY OF THE INVENTION

It is an object of the present invention to provide a high-energy implantable power source that avoids the necessity for reforming the conventional electrolytic capacitors.

It is a further object of the invention to avoid the absorption of energy commonly found in the electrolyte of the conventionally used electrolytic capacitors.

It is a further object of the invention to provide a high-energy implantable power source that does not require the use of flyback high-voltage step-up.

It is a further object of the invention to provide a high-energy implantable power source with no voltage delay.

It is a further object of the invention to provide a high-energy implantable power source with minimal relatively inconsequential internal self-discharge.

It is a further object of the invention is to provide a high-energy implantable power source that minimizes pain and trauma to the patient during episodes of defibrillation.

It is a further object of the invention to provide a high-energy implantable power source that is directly capable of delivering the voltage and energy required for stimulation of muscle and nerve tissue.

It is a further object of the invention to provide a high-energy implantable power source that may be manufactured in many and varied form factors in order to optimize its packaging with other subsystems that are part of an implantable medical device.

It is a further object of the invention to provide energy storage density per unit volume that is vastly superior to electrolytic capacitors as presently practiced.

It is a further object of the invention to provide a high-energy implantable power source that provides energy storage efficiency that is superior to electrolytic capacitors as presently practiced.

The foregoing and other objects are achieved by a high-energy battery power source with low internal self-discharge for implantable medical use. In disclosed embodiments of the invention, the power source includes a multiplicity of rechargeable energy storage battery cells, a primary power source adapted to charge the energy storage cells, a switching system adapted to switch the energy storage cells between a parallel connection configuration for charging and a series connection configuration for discharging, and circuitry adapted to initiate charging of the energy storage cells by the primary power source, but only in response to an input signifying a need to discharge energy, and to refrain from charging the energy storage cells until the input is received. In this way, the energy storage cells are maintained in a low charge state until discharge energy is required, the low charge state being at a level that promotes acceptably low internal self-discharge of the energy storage cells.

The invention further contemplates a method for providing high-energy stimulus to living tissue. When a need for high-energy stimulus is sensed, a multiplicity of rechargeable energy storage battery cells are charged from a first relatively low charge state to a second relatively high charge state. The energy storage cells are discharged following charging. The energy storage cells are maintained in the low charge state between discharges, at a charge level that produces acceptable levels of internal self-discharge.

### BRIEF DESCRIPTION OF THE DRAWINGS

FIG. 1 is a functional block diagram showing a first exemplary embodiment of the invention in which a primary high-energy density battery charges a bank of thirty rechargeable secondary battery cells.

FIG. 2 is a functional block diagram showing a second exemplary embodiment of the invention in which the single primary battery of FIG. 1 is replaced by two series-connected primary batteries.

### DETAILED DESCRIPTION OF PREFERRED EMBODIMENTS

### 1. Introduction

As discussed by way of background above, the conventional defibrillator/ICD utilizes an energy storage system comprising one or more electrolytic capacitors with a total voltage of 700 to 800 Volts. When such a system is discharged, the waveform consists of a very high peak initial voltage of 700 to 800 Volts, which falls rapidly on an exponential curve. Thus the energy delivered has an average value that is far less than the peak voltage at the beginning of the exponential fall. Dr. Werner Irnich, in a paper entitled “The Fundamental Law of Electrostimulation and its Application to Defibrillation,” PACE vol. 13, part I, pp. 1433-1447 (November 1990), has suggested that a rectangular (orthogonal) wave form should be superior to an exponential wave form for defibrillation purposes.

The high-energy battery power source disclosed herein utilizes a bank of rechargeable battery cells as energy storage elements. The discharge voltage is generated by the movement of ions from a battery anode to the battery cathode. This is a gradual process that results in a roughly rectangular voltage waveform rather than an exponential waveform. Thus, the same discharge energy will be delivered at a far lower peak voltage than with a typical capacitor discharge system. The output voltage level of the power source will be determined by the number of series-connected energy storage cells necessary to achieve that voltage. Whereas a capacitor discharge system would require a stored voltage of 700 to 800 volts, the discharge voltage from the energy storage cells taught herein is a small fraction of that, e.g., approximately 120 volts. If 4-volt lithium ion cells are used to implement the energy storage cells, this means that an power source of the type we teach herein would require only 30 energy storage cells to provide an energy discharge of 30 joules at 120 volts. Persons skilled in the art will appreciate that other combinations of voltage, current, and time will be possible wherein a discharge energy of 30 joules is be equaled or exceeded.

### II. First Exemplary Embodiment

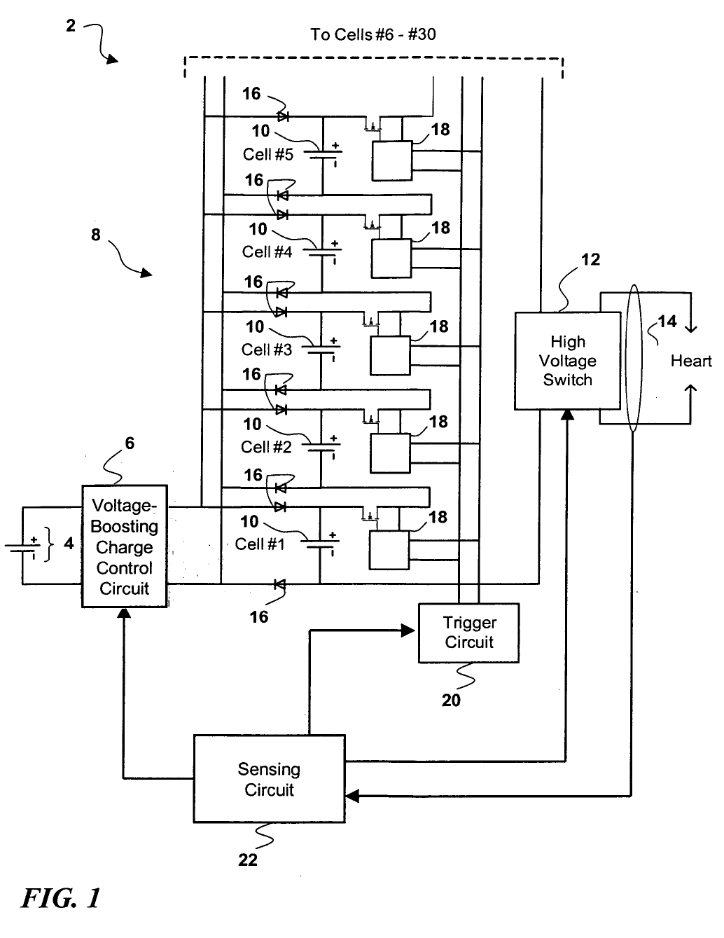

Turning now to FIG. 1, a first exemplary embodiment of the invention is illustrated by a high-energy battery power source 2 for use in an implantable device such as a defibrillators or ICD. The power source 2 includes a primary section comprising a high-energy primary battery 4 and a conventional charge control circuit 6 with voltage boost capability. The power source 2 further includes a secondary section comprising a bank 8 of multiple secondary energy storage cells 10 arranged to allow charging in a parallel circuit configuration and discharging in a series circuit configuration. The high voltage output of the battery bank 8 is connected to a high-voltage switch 12 to control the delivery of energy to implanted tissue, such as a heart 14.

The primary battery 4 is exemplified by a high capacity 2 ampere hour cell based on a suitable chemistry that is either rechargeable or non-rechargeable. Exemplary chemistry classes include lithium iodine (L/I), lithium silver vanadium oxide (Li/SVO), and lithium manganese dioxide (Li/MnO₂). The primary battery could also be implemented as a 4-volt, high-capacity lithium ion (Li-ion) cell. However, because the lithium ion chemistry does not provide particularly high energy output, another battery configuration might be preferable. For any chemistry, the voltage boost capability of the charge control circuit 6 can be used as necessary to raise the primary battery voltage up to a voltage level required to charge the secondary cells.

The battery bank 8 is powered by the primary battery 4 and the charge control circuit 6. The energy storage cells 10 of the battery bank 8 are based on a suitable rechargeable battery chemistry, such as lithium ion (Li-ion). If desired, there may be 200 energy storage cells 10 that are charged in parallel to approximately 4.0-4.2 volts (for Li-ion cells) and discharged in series to as much as 800 volts or more. Preferably, however, only 30 energy storage cells 10 will be used and the primary battery 4 will supply energy to simultaneously charge in parallel all of the energy storage cells to approximately 4.0-4.2 volts so that they can be discharged in series at approximately 120 volts.

Associated with the energy storage cells 10 are a corresponding number of parallel channels. Each channel includes a pair of blocking diodes 16. Within each channel, one of the blocking diodes 16 is connected on one side to a positive terminal of the charge control circuit 6 and on the other side to the positive terminal of an energy storage cell 10. The other blocking diode is connected on one side to a negative terminal of the charge control circuit 6 and on the other side to the negative terminal of an energy storage cell 10. This places all of the energy storage cells 10 in a position to be charged in parallel. The energy storage cells 10 are also interconnected by FET (Field Effect Transistor) switches 18 of conventional design. A trigger circuit 20 controls the state of the switches 18 as a group. When all of the switches 18 are simultaneously closed, the energy storage cells 10 are connected in series such that the batteries will discharge into an implantable defibrillator catheter (not shown) implanted in the heart 14. Note that the high-voltage switch 12 must also be closed during discharge. When the switches 18 are open, the energy storage cells 10 will be in the parallel connected charging configuration. Charging will be initiated by the charge control circuit 6 in a manner described in more detail below.

In an exemplary implementation, the power source 2 comprises a bank of 30 lithium ion energy storage cells 10, each with a maximum voltage of approximately 4.2 volts and a storage capacity of approximately 0.073 milliampere hours. Each energy storage cell 10 is charged through its blocking diodes 16. Because each blocking diode 16 has a voltage drop of approximately 0.6 volts, the voltage boost capability of the charge control circuit 6 is required in order to provide a charging voltage of approximately 5.2-5.4 volts. The net charging voltage placed on the energy storage cells 18 will thus be approximately 4.0-4.2 volts. Use of Schottky diodes could decrease the voltage drop caused by the blocking diodes 16.

It is a known characteristic of lithium ion rechargeable cells that maximum internal self-discharge will take place when the cell is at maximum state of charge, which will be approximately 4.2 volts. Such cells are shipped from the manufacturer at a voltage of about 3.8 Volts, at which internal self-discharge is minimized to a level where the internal self-discharge is acceptably low. The effective operating range of the lithium ion cell encompasses a discharged voltage of approximately 3.0 volts up to a fully-charged voltage of approximately 4.2 volts. As described in more detail below, we teach maintaining the energy storage cells 10 at a resting voltage of approximately 3.0-3.5 volts, and then periodically charging the energy storage cells to approximately 4.0-4.2 volts with 30 joules of energy provided by the primary battery 4 and the charge control circuit 6 when the power source 2 is required to deliver high-energy therapy. The reason for doing this is to improve the efficiency of the energy storage cells 10, enable them to charge up to their full energy storage capacity at approximately 4.0-4.2 volts, and then substantially discharge the energy storage cells during defibrillation with each defibrillation shock that is delivered. At the resting voltage of approximately 3.0-3.5 volts, internal self-discharge is minimized to an inconsequential level. At the fully charged voltage of approximately 4.0-4.2 volts, some minimal self-discharge will take place, but this only occurs when the power source 2 is delivering energy to implanted tissue, perhaps only a period of several minutes each year at best. This removes internal self-discharge as a meaningful loss element. It will be appreciated that resting voltages other than approximately 3.0-3.5 volts could also be maintained on the energy storage cells 10. For example, a resting voltage anywhere within a range of approximately 2.8-3.8 volts could be selected depending on the level of internal self-discharge that is deemed to be acceptable and the voltage drop off that is anticipated during high energy discharge.

### III Second Exemplary Embodiment

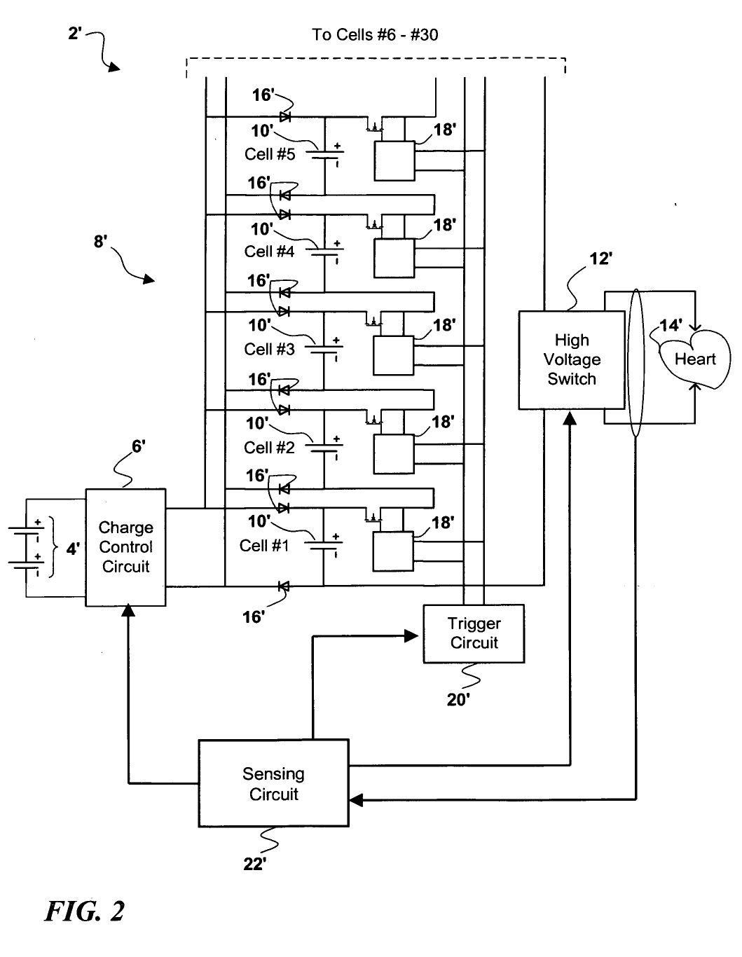

Referring now to FIG. 2, a second exemplary embodiment of the invention is illustrated by a high-energy battery power source 2′. The energy storage system 2′ is the same in all respects as the power source 2 of FIG. 1, as shown by the use of corresponding reference numerals. The only difference is that the primary battery 4 is replaced with two primary batteries 4a′ and 4b′ in a series connection, and the charge control circuit 6′ does not require voltage boost capability. By way of example only, the primary batteries 4a′ and 4b′ cells can be implemented using a battery chemistry such as lithium/carbon monofluoride (Li/CFx), with a terminal voltage of approximately 2.7 volts each. The total voltage of the series connected batteries cells 4a′ and 4b′ will thus be approximately 5.4 volts. This voltage accounts for the fact that there are two blocking diodes 16′ connected in series with each of the 30 energy storage cells 10′. Each blocking diode 16′ has forward voltage drop of approximately 0.6 volts, resulting in a total voltage drop for each energy storage cell 10′ of approximately 1.2 volts. This will reduce the voltage from the primary batteries 4a′ and 4b′ from approximately 5.4 volts down to approximately 4.2 volts, which is optimum for charging the energy storage cells 10′ if they are lithium ion cells. The second embodiment of FIG. 2 has the disadvantage of adding another battery to the power source 2′, but has the advantage of permitting each of the 30 energy storage cells 10′ to be charged without a voltage boosting inductor, thus facilitating operation with higher overall efficiency.

### IV. Operational Considerations

In an exemplary operational mode, charging of the charge storage cells 10 or 10′ will be performed upon detection of the onset of tachyarrhythmia or other therapy-triggering event. A sensing system 22 (FIG. 1) or 22′ (FIG. 2) of the type conventionally used in implantable defibrillators and ICDs can be used to provide an indication to the power source 2 or 2′ that charge/discharge cycling is required. This indication will initiate a charging response in the charge control circuit 6 or 6′. Charging will be performed for a predetermined time interval or until a predetermined charge state is reached (e.g., approximately 4.0-4.2 volts for lithium ion energy storage cells), at which time charging will be discontinued. The sensing system 22 or 22′ will then initiate discharging by triggering the switches 18 or 18′, and also triggering the high-voltage switch 12 or 12′. Discharging will be performed for a predetermined time interval or until a predetermined discharge state is reached (e.g., approximately 3.0-3.5 for lithium ion energy storage cells). The sensing system 22 or 22′ will reset the switches 18 or 18′ and the high-voltage switch 12 or 12′ to their open state. The energy storage cells 10 or 10′ will then be maintained in the low charge state (at the resting voltage) until the next energy delivery event is sensed, thereby minimizing internal self-discharge in the energy storage cells 10 or 10′ to an acceptable level.

The power sources 2 and 2′ are capable of delivering 30 joules of energy for each defibrillation shock. Assuming the energy storage cells 10 and 10′ are lithium ion cells, the charge control circuit 6 or 6′ will deliver sufficient voltage during charging to charge the energy storage cells to approximately 4.0-4.1 volts. The charging current output will be approximately 1.5 amperes for each of the power sources 2 and 2′. If charging is performed for five seconds at 4.0-4.2 volts and 1.5 amperes, approximately 30 joules of energy will be delivered to the energy storage cells 10 or 10′. As stated above, this charging current can be evenly divided to charge 30 energy storage cells 10 or 10′ in parallel.

To charge the energy storage cells 10 or 10′ to a 4.0-4.2 voltage level will require each of the energy storage cells to absorb 1/30^{th} of the energy extracted from the primary section of each power source 2 and 2′, or approximately 1 joule (1 watt second) each. If it is assumed that the energy storage cells 10 or 10′ are charged up to 4.1 volts and then discharged over to a resting voltage of 3.5 volts, the average per-cell discharge voltage is 3.8 volts. Assuming the discharge lasts for 1 second, an energy discharge level of 1 joule requires that each energy storage cell support a discharge current of approximately 0.263 amperes. Moreover, the energy storage cells 10 and 10′ should each have a capacity of about 0.263 ampere seconds or 0.073 milliampere hours. At this cell capacity, the energy storage cells 10 and 10′ will each discharge down to a point where enough energy has been removed to reach the resting voltage with one defibrillation shock. As stated, with a lithium ion cell, most of the energy will have been removed by the time the cell voltage drops to approximately 3.0-3.5 volts.

The capacity of the energy storage cells 10 and 10′ will thus be selected to meet the foregoing discharge requirements. If it is also desired to increase discharge current requirements, the number of energy storage cells 10 and 10′ can be increased for each parallel channel from a single cell up to any desired number of parallel-connected energy storage cells. In that case, all of the parallel-connected energy storage cells for a given channel would be charged in parallel and discharged in parallel within that channel. The channels themselves, each with multiple parallel connected energy storage cells, would be discharged in series in the manner described above using the switches 18 or 18′ associated with each channel.

To reduce volumetric requirements, thin-film battery constructions, such as those disclosed in U.S. Pat. Nos. 6,818,356, 6,517,968, 5,597,660, 5,569,520, 5,512,147 and 5,338,625, and in published application US2004/0018424, could be used to fabricate the energy storage cells 10 and 10′. The contents of the foregoing patents and published application are hereby incorporated herein by this reference. Performance data on thin-film energy storage cells, including lithium ion cells formed on alumina, are available at www.oakridgemicro.com/tech/tfb.htm.

Accordingly, a high-energy battery power source with low internal self-discharge for implantable medical use has been disclosed. An advantage of each disclosed system 2 and 2′ is that the energy storage cells 10 and 10′ have no significant voltage across them except during actual defibrillation. This fact reduces the internal self-discharge to insignificant levels because internal self-discharge occurs only when the energy storage cells are in a highly charged condition. Thus, the internal self-discharge that could drain the primary batteries 4 or 4a′/4b′ in a year or less becomes insignificant because the energy storage cells 10 and 10′ are charged only during defibrillation, which may total only a few minutes each year.

It will be seen that the invention accomplishes the objects set forth by way of summary above. In particular, the first object, which avoids the necessity for reforming energy storage capacitors, is achieved by the complete elimination of electrolytic capacitors from the defibrillator design.

The second object, which avoids the energy loss in electrolytic energy storage capacitors, is similarly achieved by the elimination of electrolytic capacitors from the defibrillator design.

The third object, to eliminate the energy loss in high-voltage magnetic flyback voltage converter, is achieved by the generation of the required high-voltage through the use of a multiplicity of energy storage cells, which in the case of the exemplary embodiments would be 30 lithium ion energy storage cells, charged in parallel and discharged in series to generate the defibrillation voltage of approximately 120 volts.

The fourth object, the elimination of voltage delay, can be achieved by the use of lithium ion energy storage cells, which have single stage rundown that avoids the voltage delay commonly encountered by other battery systems such as Li-SVO, which has a three step rundown pattern.

The fifth object of reducing internal self-discharge is achieved by maintaining the lithium ion energy storage cells over a voltage range of approximately 3.0 to 3.5 volts until service is required. Within this range the lithium ion energy storage cells have an internal discharge of less than 3% per year, which would permit a 50% survival at 15 years.

The sixth object, to minimize pain and trauma to the patient during the defibrillation process, is achieved by operating the power source at a voltage of approximately 120 volts rather than the 700-800 volts used in conventional implantable defibrillator/ICDs. Operation of a defibrillator/ICD at a voltage of only 15% of that of a conventional defibrillator should vastly decrease the amount of pain and trauma to which the patient is normally subjected.

The seventh object of achieving satisfactory defibrillation at a fraction of the voltage used in conventional implantable defibrillators is achieved by utilizing the rectangular waveform generated by the gradual chemical process of ion movement from anode to cathode in a battery cell rather than the extremely non-linear capacitor discharge waveform seen in conventional implantable defibrillator systems.

The eighth object, to utilize construction methods that enable assembly in various sizes and various form factors, is achieved by the use of multiple energy storage cells that are currently available in postage stamp size and have become available in solid-state formats (e.g., thin film cells), which allow far greater flexibility than previously available in conventional micro-circuitry.

The ninth object of achieving an implantable defibrillator cardioverter utilizing high-energy-density components to permit a smaller overall size is also accomplished. Most of the components in the invention taught herein (i.e., exclusive of the primary batteries) could be laid down on an automated system through vapor deposition or other processes. This will result in considerable savings in space, weight, and component count.

The tenth object, to utilize high-energy battery cells to achieve overall energy densities much higher than those available from the previously used electrolytic capacitors, is likewise accomplished.

Although specific exemplary embodiments have been shown and described, it will be apparent that various modifications, combinations and changes can be made to the disclosed designs in accordance with the invention. It should be understood, therefore, that the invention is not to be in any way limited except in accordance with the spirit of the appended claims and their equivalents.

I claim:

1. A high-energy battery power source comprising:
a multiplicity of rechargeable energy storage battery cells;
a primary power source adapted to charge said energy storage cells;
a charge control circuit adapted to control charging by said primary power source;
a switching system adapted to switch said energy storage cells between a parallel connection configuration for charging and a series connection configuration for discharging;
a sensing circuit adapted to initiate simultaneous parallel charging of said energy storage cells from a first relatively low charge state to a second relatively high charge state;
said sensing circuit being further adapted to initiate said charging only in response to an input signifying a need to discharge energy, and to refrain from initiating said charging until said input is received;
said sensing circuit being further adapted to terminate said charging at a point where said energy storage cells are in said second relatively low charge state and initiate serial discharging of said energy storage cells; and
said sensing circuit being further adapted to terminate said serial discharging at a point where said energy storage cells are in said second relatively low charge state;
whereby said energy storage cells are maintained in said first relatively low charge state until discharge energy is required, said first relatively low charge state being at a level that achieves acceptably low internal self-discharge of said energy storage cells.

2. The power source of claim 1 wherein said energy storage cells comprise a lithium ion chemistry.

3. The power source of claim 1 wherein said energy storage cells comprise a thin film construction.

4. The power source of claim 1 wherein said energy storage cells generate a voltage output of approximately 120 volts when connected in series for discharge.

5. The power source of claim 1 wherein said energy storage cells generate a voltage output of approximately 120 volts and deliver a total energy of approximately 31 joules when connected in series for discharge.

6. The power source of claim 1 wherein said energy storage cells each generate an average voltage output of approximately 3.8 volts and an energy of approximately 1 joule during discharge.

7. The power source of claim 1 wherein said energy storage cells each have a storage capacity of approximately 0.073 milliampere-hours.

8. The power source of claim 1 wherein said energy storage cells are arranged in channels each providing a current of approximately 0.263 amperes when connected in series for discharge.

9. The power source of claim 1 wherein said primary power source comprises a single battery and wherein said charge control circuit comprises a voltage boost circuit.

10. The power source of claim 1 wherein said primary power source comprises a pair of batteries.

11. A method for providing high-energy stimulus to living tissue, comprising:
sensing a need for high-energy stimulus;
in response to said sensing, charging a multiplicity of rechargeable energy storage battery cells from a first relatively low charge state to a second relatively high charge state; and
discharging said energy storage cells into said living tissue following said charging until said energy storage cells return to said first relatively low charge state.

12. The method of claim 11 wherein said charging is discontinued prior to said discharging commencing.

13. The method of claim 11 said discharging is discontinued following said energy storage cells discharging from said second relatively high charge state to said first relatively low charge state.

14. The method of claim 11 wherein said energy storage cells are charged in parallel.

15. The method of claim 11 wherein said energy storage cells are discharged in series.

16. The method of claim 11 wherein said energy storage cells are maintained in said first relatively low charge state except during said charging and discharging.

17. The method of claim 11 wherein said first relatively low charge state is selected to minimize internal self-discharge of said energy storage devices to an acceptable level.

18. The method of claim 11 wherein said charging is performed for approximately 5 seconds.

19. The method of claim 11 wherein said discharging is performed for approximately 1 second.

20. An implantable medical device, comprising:
a multiplicity of rechargeable energy storage battery cells;
a primary power source adapted to charge said energy storage cells;
a switching system adapted to switch said energy storage cells between a parallel connection configuration for charging and a series connection configuration for discharging; and
means for initiating charging of said energy storage cells only in response to an input signifying a need to discharge energy and to refrain from charging said energy storage cells until said input is received;
whereby said energy storage cells are maintained in a low charge state until discharge energy is required, said low charge state being at a level that promotes low internal self discharge of said energy storage cells.
